(19) United States
(12) Patent Application Publication
(10) Pub. No.: US 2015/0081038 A1
(43) Pub. Date: Mar. 19, 2015

(54) FEMORAL PROSTHESIS USABLE IN WATER ENVIRONMENT AND METHOD FOR MANUFACTURING SUCH PROSTHESIS

(71) Applicant: Aqualeg SAS, Nantes (FR)

(72) Inventor: Frederic Rauch, Nantes (FR)

(21) Appl. No.: 14/030,276

(22) Filed: Sep. 18, 2013

(51) Int. Cl. A61F 2/60; B32B 38/00; B32B 37/24; A61B 19/00; B32B 41/00

(52) U.S. Cl. CPC A61F 2/60; A61B 19/50; B32B 41/00; B32B 37/24; B32B 38/0004; B32B 2037/243; B32B 2255/02; B32B 2255/26
USPC 623/33; 156/64

(57) ABSTRACT

A prosthetic device for a lower limb of a user is provided which includes a femoral leg component, a foot component and a cover component which are water compatible. The cover component includes one or more hollow sleeves defining one or more cavities in communication with a cavity provided in the foot component. The foot compartment includes a hole overlapping the one or more hollow sleeves which is configured to evacuate water from the prosthetic device and to provide air communication between the one or more cavities and the exterior of the prosthetic device. The cover includes a flexible venting opening so that the water drains naturally from the one or more cavities. A method of manufacturing the prosthetic device is also provided. The method includes manufacturing a cover with successive layers forming the cavities, with the cavities configured such that water drains naturally from the prosthetic device.

### BACKGROUND OF THE INVENTION

The present invention is related to prostheses.

More particularly it concerns a prosthesis comprising covers, and covers which can be used with femoral limb prosthesis for everyday activities and water activities.

It also relates to a method of manufacturing such covers and/or prosthesis.

A typical walking leg prosthesis cannot be used in water since the components (feet, knees, adapters, foam covers) are not designed to be water resistant.

On the contrary the swim legs are disappointing for a user. They are sometimes functional, but have no cosmetic cover, and are often rudimentary both in terms of function and aesthetic.

The user is therefore not inclined to use them.

There is therefore an important need for a walking hybrid cover and/or prosthesis suitable for use in water for both an above-knee and below-knee prosthetic device. This is particularly the case for an above-knee prosthesis including an artificial knee articulation which is always involving problems in the aesthetic aspect of the leg not generally compatible with a connection of sufficient flexibility.

Commonly-owned U.S. patent application Ser. No. 13/900,999, filed May 23, 2013, and entitled “Lower Limb Prosthesis Usable in Water Environment and Method for Manufacturing Such Prosthesis,” which shares the same inventor as the instant disclosure and which is herein incorporated by reference in its entirety, describes a suitable water-compatible prosthesis for a lower limb of a user and method of manufacturing the same. The prosthesis comprises a hollow cover configured to be water compatible and to drain to an exterior of the device such that a user may wear the prosthesis when swimming, bathing, etc.

### SUMMARY OF SELECTED INVENTIVE ASPECTS

The present invention aims at solving this need by proposing a new prosthetic device which allows amputees to stroll along a beach, walk into the water, go for a swim and experience the waves just like everyone else and this at a very interesting cost and without sacrificing the aesthetic of the leg.

With the invention, the prosthesis has a cover which maintains its shape without the need for foam underneath and a knee cover that has ends maintaining their shape and with flexibility for the motion of the leg.

The covers can be used and adapted to any parts or elements of existing walking prosthesis as long as such elements are water compatible.

With the invention, it is possible for the user to wear his everyday prosthesis, bathe, have a swim, enjoy kayaking, canoeing, sailing, white water rafting, etc., and then resume his normal activities without the need to change his prosthesis.

And this is possible with a cosmetically appearing prosthesis.

Furthermore the whole device is easy to maintain as the cover can be removed from the mechanical parts of the prosthesis and reattached easily, and as the knee covers can be easily moved or manipulated for enabling access to the artificial knee.

It can also be used with different prosthetic components and change from one to another, and/or easily adjusted to do so.

The invention comes from the observation that a traditional prosthesis is buoyant or covered with foam not designed for immersion, meaning that it tends to float in the water. One of the ideas of the invention is therefore to propose a prosthesis with a cover which allows water to enter into the cover, to permit neutral buoyancy, which means that the leg will not have the tendency to float.

By achieving neutral buoyancy, the invention device can therefore be used for all types of water activities, including scuba diving.

When exiting from a pool or the ocean, etc., the device of the invention allows a quick and discreet drain through the device, permitting the user to resume his normal activity immediately.

The difficulty of giving a nice aspect to the cover knee, which has to undertake folding on its back (behind the knee), is also advantageously resolved in one embodiment, bearing in mind that the fact of adding a cover knee does not change the buoyancy properties.

To achieve this aim the invention mainly proposes a prosthetic device for a lower limb of a user comprising a femoral leg component, having a femoral part presenting a proximal end forming a socket to be connected to the stump of the user, an intermediate knee part and a tibial part having a distal end, a foot component having a foot shell defining a first cavity within said foot shell and having an open upper part, said foot component being arranged to be articulated to the distal end of the tibial part with adapters, and a cover component covering said leg component, said leg, foot and cover components being water compatible,

wherein the cover component is formed by a femoral cover part, a knee cover part and a tibial cover part,

said femoral cover part being formed by a hollow flexible first sleeve arranged to cooperate contiguously or overlappingly with the socket on one side and with the knee cover part on the other side,

said knee cover part being formed by a second sleeve defining a second cavity within said second sleeve around said knee component, said second sleeve being formed by a flexible self-carrying hollow tubular portion comprising a semi-rigid top end connected to said femoral cover part, an intermediate portion having a semi-rigid front end, and a flexible back end arranged to authorize the flexion of the knee component, and a semi-rigid bottom end connected to said tibial cover part,

and said tibial cover part formed by a hollow flexible self-carrying third sleeve defining a third cavity within said third sleeve around the tibial part and in communication with said second cavity, said tibial cover part being arranged to cooperate contiguously or overlappingly with the bottom part of the knee cover part on one side and to cooperate overlappingly with the upper part of the foot shell on the other side on a predetermined height, said first cavity being in communication with said third cavity, said foot shell comprising a hole on its inner side, said hole overlapping the third sleeve for evacuation of the water and presenting an aperture below the third sleeve to provide air communication between the cavities and the exterior of the device,

and wherein the cover component is arranged to provide in an upper part (advantageously close to the knee part), a flexible venting opening for ventilation, so that the water drains naturally from the first and second cavities.

The invention also proposes additionally and/or alternatively the following embodiments:
the semi-rigid top end of the knee cover part is slightly fittingly connected to the femoral cover part and can be rolled back for access to the knee component, such slightly fitting connection providing the flexible venting opening;
the semi rigid bottom end of the knee cover part is slightly fittingly connected to the tibial cover part and can be rolled back or adjusted for access to the knee component, such slightly fitting connection authorizing the flexible venting opening;
the knee cover part is integral with the tibial cover part;
the knee cover part presents a woven external aspect such as an orthopaedic knee cap;
the flexible back end of the knee cover part comprises an air porous flexible film, also authorizing venting;
the hole of the foot shell overlapping the sleeve of the tibial cover part is of at least one centimetre and presents an aperture below the sleeve extending until the top surface of the foot component (at the bottom of the foot shell) to provide air communication between the cavities and the exterior of the device;
the cover component comprises in its upper part, close to the socket, and to form the aperture for ventilation, a longitudinal slot comprising two contiguous lips, so that the water drains naturally from the first and second cavities. This is particularly relevant when the cover component is of one piece;
the hole of the foot shell is a rectangle of width between 0.3 mm and 1 cm and advantageously 0.5 cm and of length between 2 cm and 5 cm, for instance 4 cm, with an overlapping with the sleeve of between 0.5 cm and 2 cm, for instance 1 cm;
the slot is of a length between 2 cm and 4 cm, for instance 3 cm;
the sleeve comprises an overlay of at least three layers, at least the external layer being transparent;
the third sleeve comprises three parts, i.e.

a first part directed towards the foot which can be rolled back and which cooperates slightly fittingly with the upper part of the foot shell,

a second part which is connected to the first part with bending capabilities and which is rigid or substantially rigid along the main central part of the tibia component, and

a third part connected to the second part with bending capabilities (flexible), said third part being fixed or integral with the knee cover part;
the first and third sleeves are made of a superposition of layers, wherein at least one layer is a transparent or translucent silicone layer which forms the external layer of the sleeve, at least two layers of dyed elastic fabric, at least the elastic fabric closer to the external layer being light permeable.

Advantageously, the dyed elastic fabric layers are of two different colours.

The superposition of a transparent silicone layer with a layer of elastic fabric permits the mechanical resistance of the sleeve and gives the needed rigidity while authorizing, by modification of the thickness of the layers at different positions along the sleeve, its bending capacities.

The superposition of two layers of elastic fabric authorizes to modify the permeability to the light, which permits a great number of combinations while obtaining a particularly aesthetic result, mimicking particularly well real skin.

In another advantageous embodiment, the sleeve comprises two layers of silicone sandwiching at least two layers of elastic fabric.

Advantageously the second layer of silicone is opaque.

Advantageously the sleeves further comprise at least a layer of painting between two layers of elastic fabric, for instance made of polyamide, having for instance between 1.10³ to 3, 33 10⁻³ g/m of fabric wires, preferably between 1, 33×10⁻³ g/m and 2, 77×10⁻³ g/m.

In an advantageous embodiment the semi rigid parts of the second sleeve comprise a superposition of a first internal layer of silicone or an elastomer with a thickness providing the relevant rigidity according to what is appreciated by one skilled in the art, and an external layer of a fiber elastic tissue (said elastic fibers being for instance impregnated by the silicone) or textile.

The invention also relates to a method for manufacturing covers for lower leg prosthesis as described here above.

It also concerns a method for manufacturing a cover component for leg prosthesis of a user in three parts, i.e. a femoral cover part, a knee cover part and a tibial cover part, comprising the steps of:
taking digital pictures with a calibration pattern of the healthy limb (the sound limb) of a user and/or through a scanner allowing such calibration according to four directions (rear, front and lateral faces) such as the tibial and/or femoral parts of the healthy leg is precisely determined,
transmitting and/or incorporating such digital pictures to a distal server via Internet together with references and specification of a leg and a foot components determined by the prosthesis maker of the user, (for instance obtained by scanning the prosthesis)
digitally reconstructing by symmetry (superposition) the external geometry of the missing leg of the user,
designing by CADCAD the cover component taking the leg and foot components specifications into account, said leg component having an upper socket to be connected to the stump of the user and said foot component having a hollow foot shell,
forming a plain model of said cover component with a robotic milling apparatus (for instance in plastic material such as polystyrene)
manufacturing the three parts of the cover component, using the parts of said model as two or three supports, by forming or depositing successive lay outs of plastic and/or textile on said supports, in order to obtain multiple plastic and/or textile layers and therefore forming two or three sleeves (separate first, second and third sleeves or first sleeve and second and third sleeves integral together) defining cavities,
peeling or extracting the sleeves from their supports,
cutting in the foot shell a hole of at least 0.5 cm of width on its inner side, said hole being placed in a position such as the sleeve overlaps said hole on an height of a predetermined value and presents an aperture below the sleeve and towards the bottom of the foot shell when in place on an height of at least a second predetermined value and,
opening a hole in the upper part of the cover, and/or slightly fittingly adapting the second sleeve to the first and/or the third sleeve, so that the water drains naturally from the cavities of the cover and leg components.

Advantageously the second sleeve is fixed to the third sleeve.

Also advantageously the opening of the cover component is undertaken after putting in place said cover component on the leg and foot components.

In an advantageous embodiment, the manufacturing of the first and third sleeves comprises the following steps:

(a) putting an elastic fabric casing jacket on the corresponding part of the plain model to form a first elastic fabric layer,

(b) covering said casing jacket with a layer of silicone,

(c) repeating steps (a) at least twice and step (b) at least once for forming each of said sleeves, for obtaining at least a superposition of layers as follows, from the external part of the sleeve towards the internal part, a transparent or translucent silicone layer, at least two elastic fabric layers, one silicone layer and at least one elastic fabric layer.

Also in one embodiment the invention provides the following steps:

The knee cover part is rigidified to form a semi-rigid top end connectable to said femoral cover part, and a semi-rigid bottom end connectable or integral with the tibial cover part, and to form an intermediate portion having a semi-rigid front end and a flexible back.

Rigidity is the aptitude of an object to oppose elastic deformation. It is proportional to the Young's modulus and to the moment of inertia of the object (depending on its shape).

Here, by semi-rigid, one should understand what one skilled in the art understands in the field of flexible sleeves provided for injured limbs.

Semi-rigid means a Young's modulus of the material with a hollow shape as the tubular shape and regular dimensions of the cover components of the invention, which will authorize self-support of the cover, but which also comprises flexibility with an adequate shape memory in a way which will be easily determined by one skilled in the art.

The invention will be better understood by referring to the following description of embodiments given without limitation by way of example.

The description is related to the accompanying drawings, in which:

### BRIEF DESCRIPTION OF THE DRAWINGS

FIG. 1 is a first lateral view of the embodiment of the invention more particularly described here.

FIG. 2 is a second lateral view of a second embodiment of the invention.

FIG. 3 is a lateral view of the foot component with its foot shell, according to an embodiment of the invention.

FIG. 4 is a section view along IV-IV of the prosthesis of FIG. 2.

FIG. 5 is a section view along V-V of the prosthesis of FIG. 2.

FIG. 6 illustrate an external view of the knee cover part of the embodiment of FIG. 2, when bended.

FIG. 7 shows separately the components of the embodiment of FIG. 1.

FIG. 8 shows schematically the different steps of the method of the invention as more particularly described here.

### DETAILED DESCRIPTION OF EXAMPLE EMBODIMENTS

Hereinafter in the description, the same reference numbers will be used to designate the same elements.

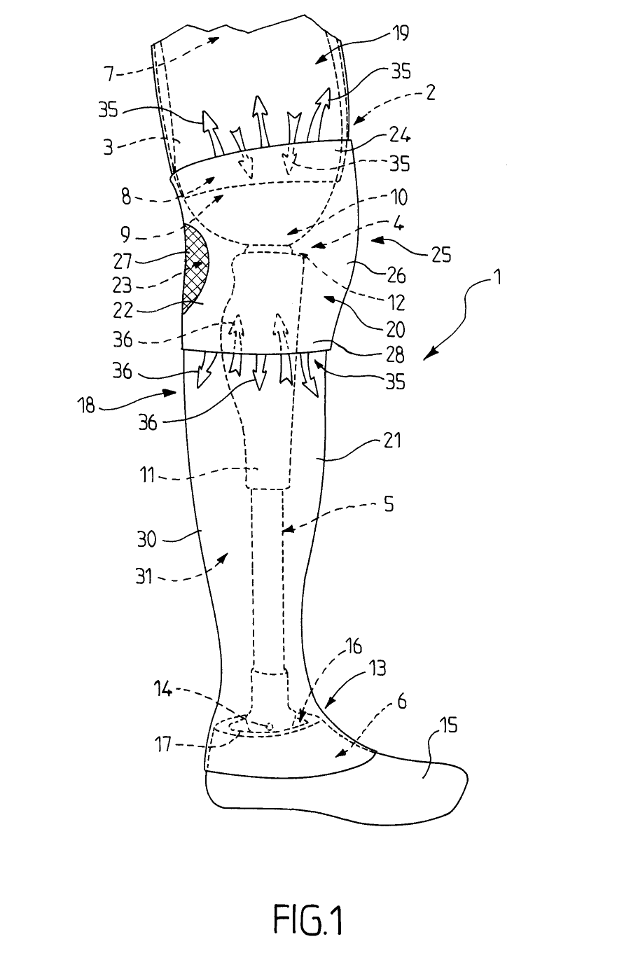

FIG. 1 shows a first embodiment of a prosthetic device 1 for lower limb of a user comprising a femoral leg component 2 comprising from top to bottom, a femoral part 3, an intermediate knee part 4, a tibial part 5 and a foot component 6.

The femoral part 3 has a proximal (top) end 7 forming a socket to be connected to the stump of the user (not shown), said socket being connected through its distal end 9 to the intermediate knee part 4.

The intermediate knee part 4 is connected by its proximal end 10 to the articulate portion 11 of the knee part 4 (prosthetic knee) by adapters 12, known per se, said articulate portion 11 being connected on the other side to the tibial part 5 having a distal end presenting an adapter 14, also known per se.

The foot component 6 is articulated to the distal end 13 of the pylon by said adapter.

It comprises a foot shell 15 defining a first cavity 16 with an open upper part 17.

The device comprises additionally a cover component 18 covering the femoral leg component.

More precisely, the cover component 18 is formed by a femoral cover part 19 connected to a knee cover part 20 that is connected to a tibial cover part 21.

In the embodiment of FIG. 1 the cover component is in three self-carrying pieces.

The femoral cover part 19 is formed by a hollow flexible first sleeve arranged to cooperate contiguously and/or through overlapping (or underlapping) tight fitting junction with the socket on one side and with the knee cover part 20 on the other side.

The connection between the femoral cover part 19 and the knee cover part 20 is tight fitting but authorizes the ventilation of the interior of the cover parts as it is not fixed on it and slidably cooperates during function and bending of the prosthesis.

The knee cover part 20 is formed by a second sleeve 22 defining a second cavity 23 within said second sleeve 22 around the intermediate knee part 4.

The semi rigid top end of the knee cover part is slightly fittingly connected to the femoral cover part and can be rolled back for access to the knee component.

The second sleeve is formed by a flexible self-carrying hollow tubular portion comprising a semi-rigid top end 24 connected to the femoral cover part 19, an intermediate portion 25 having a semi-rigid front end 26, and a flexible back end 27 arranged to authorize the flexion of the knee part 4, and a semi-rigid bottom end 28 connected to the tibial cover part.

In the present example the knee cover part is held slightly fittingly while allowing venting (i.e., communication of air between the inside of the cover and the outside of the cover), at its two ends by the femoral cover and the tibial cover parts, but advantageously it is fixed on the tibial cover part, therefore authorizing the sliding of the knee part only on the femoral cover part.

Due to the material used, i.e. silicone for instance friction is sufficient to maintain the connection while allowing venting.

The flexible back end 27 allows the flexion, i.e. the articulation of the femoral cover (together with the one generated by the knee part) with an acceptable maximum angle of flexion, i.e. for instance 90°.

The rigidity is sufficient so that there is no deformation of the circumference of the bonded parts (top and bottom ends of the knee cover part), that is to say that there is substantially the same diameter regardless of the flexion.

In one embodiment the knee cover part is self-carrying and/or the knee cover part is fixed, by glue or any suitable means, and/or integral with the tibial cover part.

In another embodiment, the femoral cover part 19 is integral with the knee cover part 20, which is integral with the tibial cover part 21, i.e. all the sleeves are formed integrally together with different thickness and material which may then be used.

In such embodiment, the elements of the knee cover part (i.e. semi-rigid bottom and top end, and flexible medium part) are within the whole cover component which gives the aspect of a complete normal leg contrarily to the embodiment of FIG. 1, using misdirection on the infirmity of the user, due to the use of what seems to be a simple knee cap.

Here the knee cover part is for instance fixed to the top part of the tibial cover part and presents a woven external aspect such as an orthopaedic knee cap (FIG. 1).

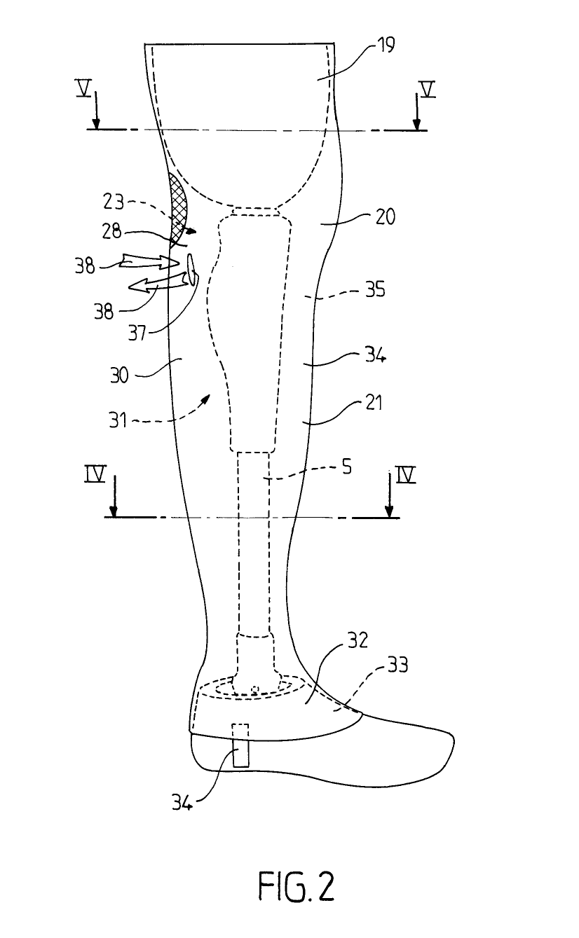

The tibial cover part will be now more precisely described in reference to FIGS. 1 and 2.

As already indicated, the tibial cover part is formed by a hollow flexible self-carrying third sleeve 30 defining a third cavity 31 within said third sleeve around the tibial part 5.

More particularly referring to FIG. 2, the third sleeve 30 comprises three parts, a first part 32 directed towards the foot which can be rolled back and which slightly fittingly cooperates with the upper part 33 of the foot shell, a second part 34 which is connected to the first part with bending capabilities and which is rigid or substantially rigid along the main central part of the leg part 5 and a third part 35 connected to the second part with bending capabilities, said third part being fixed or integral with the bottom end 23 of the knee cover part 20.

The third cavity 31 is in communication with the second cavity 23.

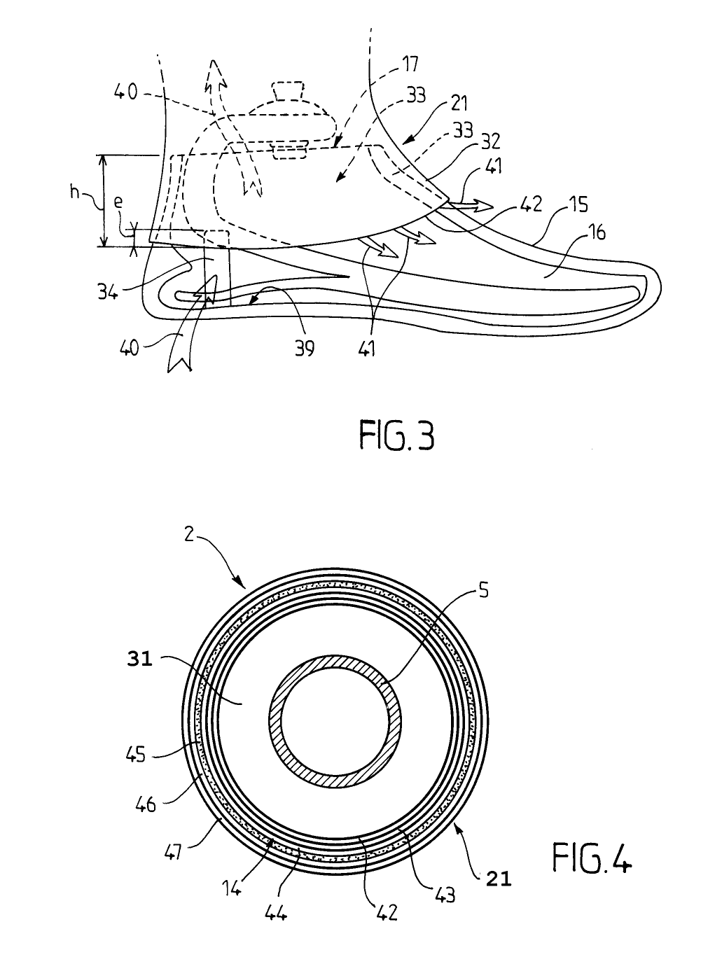

Now and more particularly in view of FIG. 3, the bottom or first part 32 of tibial cover part 21 is arranged to cooperate contiguously with the upper part 33 of the foot shell on a predetermined height h.

The first cavity 16 is in communication with the third cavity.

The foot shell 15 comprises a hole 34 on its inner side. The hole is overlapping the tibial sleeve for evacuation of the water and also present an aperture below the tibial sleeve to provide air communication between the cavities and the exterior of the device.

In order to provide ventilation for the hollow tube formed by the connecting sleeves, the cover component is configured to allow air to enter and/or exit the tube through the connection of the sleeves (which is tight but not airtight) (see arrows 35 and/or 36 on FIG. 1) and/or the cover component comprises an aperture 37 in its upper part (see arrows 38 in FIG. 2).

In the embodiment of FIG. 2 for instance the tibial cover part component additionally comprises in its upper part, close to the knee cover part junction 28, a longitudinal slot 37 comprising two contiguous lips for ventilation so that the water drain naturally and immediately from the first and second cavities, when the user get out of the water.

Referring again to FIG. 3 the tibial cover part cooperates with the upper part 33 of the foot shell 15 on the other side, substantially on a predetermined height h.

The foot shell 15 comprises a hole 34 making the interior of the first cavity 16 communicate with the exterior of the device, and situated on his inner side.

The hole is overlapping the lower part of the tibial sleeve by at least a distance e of one cm and presents an aperture below the tibial sleeve extending until the top 39 of the surface of the foot component to provide air communication between the cavities and the exterior (arrows 40).

Some leaks (arrows 41) will also exist on the periphery of the lower part 42.

FIG. 4 shows a cross section view along IV-IV of the tibial cover part (and/or of the femoral cover part) of the embodiment of the invention more particularly described here.

The tibial sleeve comprises an internal layer 42 in elastic fabric such as tubular nyglas from the company Otto Bock reference 623T9 and then, from the internal part of the tibial sleeve towards the external part, an opaque silicone layer 43, an elastic dyed fabric layer 44, a (misty) layer of paint 45, an elastic dyed layer 46 permeable to the light, and en external transparent silicone layer 47, each layer being of thickness corresponding to the requested rigid or bending (flexible) qualities of the tibial sleeve at its different parts.

At least one layer is a transparent or translucent silicone layer which forms the external layer of the sleeve, at least two layers are of dyed elastic fabric and at least the elastic fabric closer to the external layer being light permeable.

In one embodiment, at least one of the first and third sleeves comprises two layers of silicone sandwiching at least two layers of elastic fabric. The second layer of silicone is opaque. The sleeve further comprises at least a layer of painting between two layers of elastic fabric.

FIG. 5 shows a cross section along V-V of the first sleeve (femoral) showing the interior of the sleeve i.e. the femoral cover part 19, the knee cover part 20, and the tibial cover part 21.

Concerning the knee cover part, it comprises the textile flexible portion 27, the semi-rigid top end 24, the semi-rigid bottom end 28, and the semi-rigid front end 26.

The femoral cover part 19 is tubular, with a substantially oval cross-section which tapers towards the top.

The portion intended to be in contact with the interior of the stump is less high that the portion diametrically opposed.

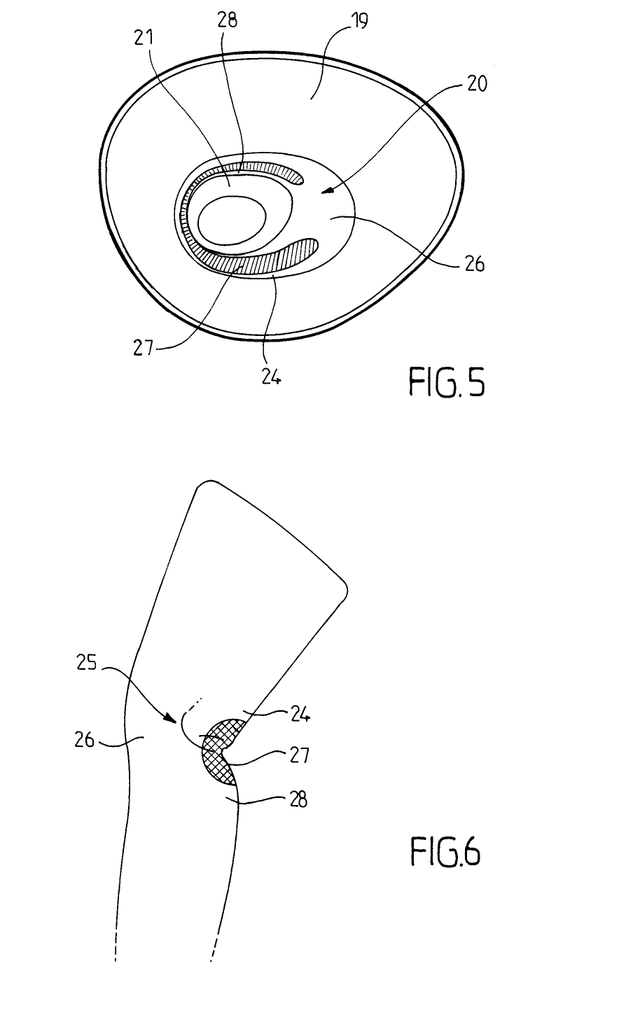

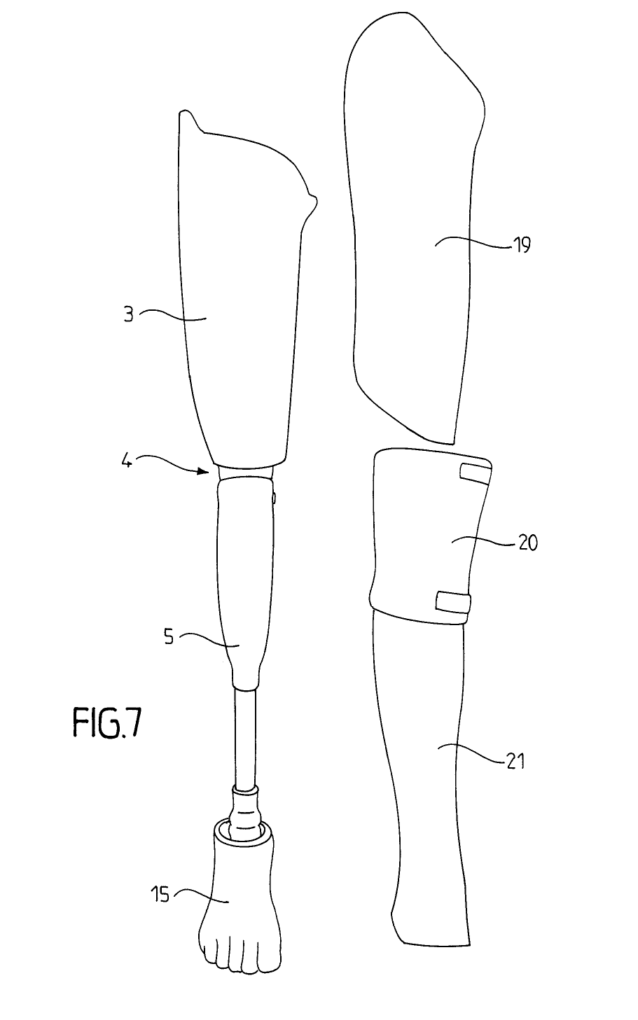

FIG. 6 shows a lateral view of the prosthesis, slightly bended.

FIG. 7 shows in exploded view the different elements comprising the prosthetic device of FIG. 1. It appears clearly that the device is entirely separable in piece, which will be easy to manipulate and install not mentioning their manufacturing also easy to operate.

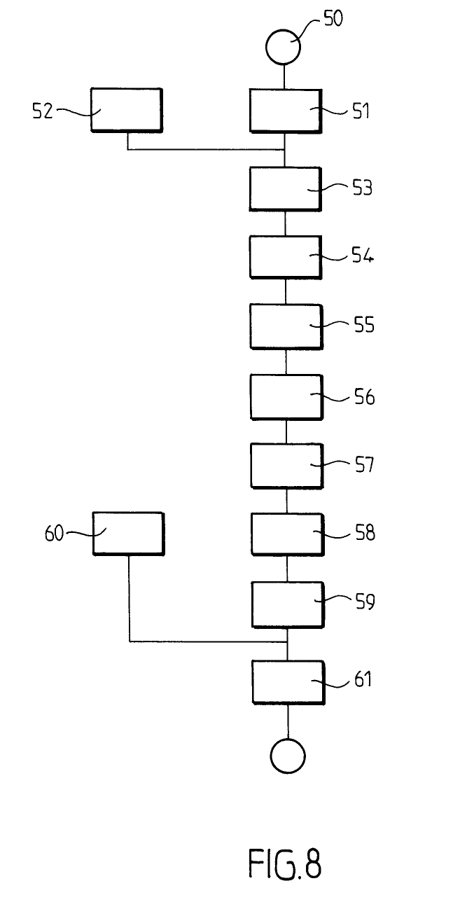

A method for manufacturing a cover for leg prosthesis of a user according to one embodiment of the invention, will be now described in particular referring to FIG. 8.

The method comprises a first step (50) of acquiring digital images such as picture of the healthy limb of a user (50). This can be done by taking digital pictures with a calibration pattern of the healthy limb and/or through a scanner allowing such calibration according to four directions (rear, front and lateral faces) such as the tibial, knee and femoral parts of the healthy leg is precisely determined.

The acquired digital data (pictures) are then transmitted and/or directly incorporated to a distal server (51). This communication can be made via a network as for instance Internet.

The data are transmitted and/or incorporated together with references and specification of a leg and a foot components determined by a third person like a prosthesis maker of the user (52).

The external geometry of the missing leg of the user is then digitally reconstructed (53). This reconstruction is done particularly by symmetry (superposition) compared to the healthy leg.

The cover components taking the leg and foot components specifications into account are designed by CAD CAD (step 54) using known software such as the software known as Solid Works by the US firm Solid Works.

An upper socket to be connected to the stump of the user is provided (step 55) to the leg component and said foot component has a hollow foot shell.

A plain model of said cover components is formed in 56. For example, this is done with a robotic milling apparatus.

The plain model will be a support for the sleeves to manufacture.

The following step (57) is to produce the sleeve by forming or depositing successively on the support, layers of elastic fabric, silicone and/or painting and/or textile, for instance in successive operations provided automatically and/or manually on the supports, in order to obtain a multiple silicone and/or textile layers forming sleeves defining cavities.

This step of manufacturing of the sleeves can be achieved by putting an elastic fabric casing jacket on the plain model to form a first elastic fabric layer.

Then the casing jacket is covered with a layer of silicone.

After that a new elastic fabric layer should be put on the casing jacket.

The steps for manufacturing of the sleeves are repeated for forming the sleeves, for obtaining at least a superposition of layers as follows, form the external part of sleeve, towards the internal part, a transparent or translucent silicone layer, at least two elastic fabric layers, one silicone layer and at least one elastic fabric layer.

In one embodiment of the invention the knee cover part is rigidified in 58 to form a rigid top end.

The semi-rigid top end is connectable to the femoral cover part.

The knee cover part has also a semi-rigid bottom end. The bottom end is connectable or integral with the tibial cover part.

The knee cover part forms an intermediate portion having a semi-rigid front end and a flexible back.

By semi-rigid, it is again to be understood a product with a sufficient flexibility but which comes back naturally in its normal position through mechanical shape memory reaction.

When the sleeves are finished, it can be kept for drying and then extracted or peeled from its support and prepared (deburred) (step 59).

If needed a hole is made in the foot (step 60) by cutting in the foot shell a hole of at least 0.5 cm of width on its inner side. The hole is placed in a position such as the sleeve overlaps said hole on an height of at least 1 cm and presents an aperture below the tibial sleeve and towards the bottom of the foot shell when in place on an height of at least 2 cm.

In an embodiment of the invention, such as for instance those where the first, second and third sleeves are integral, there is a step (61) of cutting a slot comprising two contiguous lips in the upper part of the cover component, so that the water drains naturally from the cavities of the cover and leg components.

In another embodiment, particularly those where the knee cover part is not integral with the tibial cover part, there is no need for a hole except to optimize the water flow.

The cutting steps can be undertaken after having put in place the cover component on the leg and foot components.

The assembly of the prosthesis usable in water environment will now be described.

The femoral, knee, tibial leg components are assembled.

Then, and in the same order, the femoral, knee and tibial covers are assembled to the corresponding leg component by sliding the leg component from the tibial end.

Parting from the sleeve of the cover member thus formed and after assembling the foot member on the leg member together, the femoral sleeve is prepared by returning the (soft) upper part of the sleeve which is put upside down and introduced on the socket of the leg member.

Then the combined knee and tibial cover parts are put on the tibial component with the top end of the knee cover part returned. Then this top end is slid back on the femoral sleeve.

The lower part of the tibial sleeve is then returned (here again thanks to its bendable or flexible character) and put into place on the foot upper part to be jointly adapted to it.

The upper part of the femoral sleeve is then put in place to obtain the prosthesis according to the embodiment of the invention more particularly described here.

All materials (layers, fasteners, adhesives, elastics . . . ) and in particular those forming the cover are water compatible.

As goes without saying and as is in any case obvious from the foregoing, the present invention is not restricted to the embodiments more particularly described. On the contrary, it encompasses all variants thereof particularly those in which the layers of material are different but with the same characteristic.

1. A prosthetic device for lower limb of a user comprising a femoral leg component, having a femoral part presenting a proximal end forming a socket to be connected to the stump of the user, an intermediate knee part and a tibial part having a distal end, a foot component having a foot shell defining a first cavity within said foot shell and having an open upper part, said foot component being arranged to be articulated to the distal end of the tibial part with adapters, and a cover component covering said leg component, said leg, foot and cover components being water compatible,
wherein the cover component is formed by a femoral cover part, a knee cover part and a tibial cover part,
said femoral cover part being formed by a hollow flexible first sleeve arranged to cooperate contiguously or overlappingly with the socket on one side and with the knee cover part on the other side,
said knee cover part being formed by a second sleeve defining a second cavity within said second sleeve around said knee component, said second sleeve being formed by a flexible self-carrying hollow tubular portion comprising a semi-rigid top end connected to said femoral cover part, an intermediate portion having a semi-rigid front end, and a flexible back end arranged to authorize the flexion of the knee component, and a semi-rigid bottom end connected to said tibial cover part,
and said tibial cover part formed by a hollow flexible self-carrying third sleeve defining a third cavity within said third sleeve around the tibial part and in communication with said second cavity, said tibial cover part being arranged to cooperate contiguously or overlappingly with the bottom part of the knee cover part on one side and to cooperate overlappingly with the upper part of the foot shell on the other side on a predetermined height, said first cavity being in communication with said third cavity, said foot shell comprising a hole on its inner side, said hole overlapping the third sleeve for evacuation of the water and presenting an aperture below the third sleeve to provide air communication between the cavities and the exterior of the device,
and wherein the cover component is arranged to provide in an upper part a flexible venting opening for ventilation, so that the water drains naturally from the first and second cavities.

2. A prosthetic device according to claim 1, wherein the semi-rigid top end of the knee cover part is slightly fittingly connected to the femoral cover part and can be rolled back or adjusted for access to the knee component, such slightly fitting connection providing the flexible venting opening.

3. A prosthetic device according to claim 1, wherein the knee cover part is integral with the tibial cover part.

4. A prosthetic device according to claim 1, wherein the semi-rigid bottom end of the knee cover part is slightly fittingly connected to the tibial cover part and can be rolled back or adjusted for access to the knee component.

5. A prosthetic device according to claim 1, wherein the knee cover part presents a woven external aspect such as an orthopaedic knee cap.

6. A prosthetic device according to claim 1, wherein the flexible back end of the intermediate portion comprises an air porous flexible film, authorizing venting.

7. A prosthetic device according to claim 1, wherein the third sleeve comprises three parts, a first part directed towards the foot which can be rolled back and which slightly fittingly cooperates with the upper part of the foot shell, a second part which is connected to the first part with bending capabilities and which is rigid or substantially rigid along the main central part of the leg component and a third part connected to the second part with bending capabilities, said third part being fixed or integral with the bottom end of the knee cover part.

8. A prosthetic device according to claim 1, wherein the hole of the foot shell overlapping the sleeve of the tibial cover part is of at least one centimetre and presents an aperture below the sleeve extending until the top surface of the foot component to provide said air communication between the cavities.

9. A prosthetic device according to claim 1, wherein the first sleeve and the third sleeve are made of a superposition of layers, wherein at least one layer is a transparent or translucent silicone layer which forms the external layer of the sleeve, at least two layers are of dyed elastic fabric and at least the elastic fabric closer to the external layer being light permeable.

10. A prosthetic device according to claim 9, wherein the sleeves comprise two layers of silicone sandwiching at least two layers of elastic fabric.

11. A prosthetic device according to claim 9, wherein the second layer of silicone is opaque.

12. A prosthetic device according to claim 9, wherein the sleeve further comprises at least a layer of painting between two layers of elastic fabric.

13. A prosthetic device according to claim 9, wherein the second sleeve is integral with the third sleeve.

14. A prosthetic device according to claim 9, wherein the second sleeve presents an external woven aspect such as an orthopaedic knee cap.

15. A prosthetic device according to claim 1, wherein the semi-rigid of the second sleeve comprises a superposition of a first internal layer of silicone or an elastomer and an external layer of a fiber elastic tissue.

16. A method for manufacturing a cover for leg prosthesis of a user comprising the steps of:
taking digital pictures with a calibration pattern of the healthy limb of a user and/or through a scanner allowing such calibration according to four directions (rear, front and lateral faces) such as the tibial, knee and femoral parts of the healthy leg is precisely determined,
transmitting and/or incorporating such digital pictures to a distal server via Internet together with references and specification of a leg and a foot components determined by the prosthesis maker of the user,
digitally reconstructing by symmetry (superposition) the external geometry of the missing leg of the user,
designing by CAD CAD the cover components taking the leg and foot components specifications into account, said leg component having an upper socket to be connected to the stump of the user and said foot component having a hollow foot shell,
forming a plain model of said cover components with a robotic milling apparatus,
manufacturing the three parts of the cover component using said model as supports, by depositing or forming successive lay outs of silicone and/or textile on said supports, in order to obtain a multiple silicone and/or textile layers forming sleeves defining cavities,
peeling or extracting the sleeves from the supports,
cutting in the foot shell a hole of at least 0.5 cm of width on its inner side, said hole being placed in a position such as the sleeve overlaps said hole on an height of at least one centimetre and presents an aperture below the sleeve and towards the bottom of the foot shell when in place on an height of at least two centimetres and,
opening a hole in the upper part of the cover component and/or slightly fittingly adapting the second sleeve to the first and/or the third sleeve, so that the water drains naturally from the cavities of the cover and leg components.

17. A prosthetic device according to claim 16, wherein the second sleeve is fixed to the third sleeve.

18. A method according to claim 16, wherein the opening is undertaken after having put in place the cover component on the leg and foot components.

19. A method according to claim 16, wherein the manufacturing of the first and third sleeves comprises the following steps:
(a) putting an elastic fabric casing jacket on the plain model to form a first elastic fabric layer,
(b) covering said casing jacket with a layer of silicone,
(c) repeating steps (a) at least twice and step (b) at least once for forming said sleeves, for obtaining at least a superposition of layers as follows, form the external part of sleeve, towards the internal part, a transparent or translucent silicone layer, at least two elastic fabric layers, one silicone layer and at least one elastic fabric layer.

20. A method according to claim 16 wherein the knee cover part is rigidified to form a semi-rigid top end connectable to said femoral cover part, and a semi-rigid bottom end connectable or integral with the tibial cover part, and to form an intermediate portion having a semi-rigid front end and a flexible back.
